(19) United States
(12) Patent Application Publication
(10) Pub. No.: US 2017/0014515 A1
(43) Pub. Date: Jan. 19, 2017

(54) Implantable Polymeric Device for Sustained Release of Sufentanil

(71) Applicant: ANTECIP BIOVENTURES II LLC, New York, NY (US)

(72) Inventor: Herriot Tabuteau, New York, NY (US)

(21) Appl. No.: 15/238,614

(22) Filed: Aug. 16, 2016

Related U.S. Application Data
(63) Continuation of application No. 13/468,215, filed on May 10, 2012, now abandoned.
(60) Provisional application No. 61/484,222, filed on May 10, 2011.

(51) Int. Cl. A61K 47/34; A61K 9/00; A61K 31/4535

(52) U.S. Cl. CPC A61K 47/34; A61K 31/4535; A61K 9/0024

(57) ABSTRACT

Extended release of sufentanil is provided by implanting a device containing sufentanil into a mammal or human being. The device may include a solid biocompatible polymer matrix, and sufentanil may be encapsulated within the polymer matrix. The polymer matrix comprises a plurality of pores configured to allow contact between the sufentanil and a physiological fluid of a mammal into which the device is implanted to thereby release sufentanil in vivo from the device into the mammal. The devices may be used to treat opioid addiction or pain.

### CROSS-REFERENCE TO RELATED APPLICATIONS

This application is a continuation of U.S. application Ser. No. 13/468,215, filed on May 10, 2012, which claims the benefit of U.S. Provisional Application No. 61/484,222, filed on May 10, 2011, which is incorporated by reference herein in its entirety.

### BACKGROUND OF THE INVENTION

Sufentanil is a very potent opioid. Some have reported sufentanil to be about 5 to 10 times as potent as fentanyl or 500 to 1,000 times as potent as morphine. It also has a much higher therapeutic index than fentanyl and morphine. These qualities, greater potency and potentially better safety, make it an attractive compound for clinical use. However its role has been largely restricted to the hospital setting since it is not orally bioavailable and so must be given parenterally (e.g. intravenously and intrathecally). In addition, since it has a relatively short half-life, it is not suitable for long term use.

Many patients with chronic pain require long-term continuous dosing with opioid analgesics. Effective treatment often necessitates the ingestion of multiple tablets per day. This situation can lead to poor compliance, inadequate pain relief and the development of addiction or dependence due to the peak and troughs in drug serum levels, and the potential for drug diversion for illicit use. In addition, patients on long-term oral therapy are subject other opioid-related side effects.

Addiction to heroin and other opioids has traditionally been treated with methadone, and more recently buprenorphine. Opioids with long plasma half-lives and that are orally or sublingually available (e.g. methadone and buprenorphine) have generally been used to treat opioid addiction. Because opioids such as methadone and buprenorphine are given orally or sublingually, they can be diverted for illicit use. In addition, compliance can be less than ideal. Sufentanil has not been used for the treatment of opioid dependence as it does not have a long plasma half-life and is not usually orally administered.

Sufentanil's potency and potential safety make it attractive for the treatment of chronic pain and opioid addiction and dependence if it could be delivered continuously outside of the hospital in a convenient way without the need for intravenous administration and its attendant costs and inconveniences. Thus, there is a need for a sufentanil formulation that will allow the drug to be used outside of the hospital setting for long-term continuous administration, and that will improve compliance, improve safety and reduce the potential for abuse and diversion.

### SUMMARY

Devices comprising an opioid such as sufentanil and a polymeric matrix may allow sufentanil to be used for the treatment of pain or opioid addiction by maintaining a therapeutic level of sufentanil through extended release of the compound. The devices may also improve compliance and reduce the potential for diversion.

Some embodiments include an implantable device comprising: a solid biocompatible polymer matrix; and sufentanil encapsulated within the polymer matrix. The polymer matrix may comprise a plurality of pores configured to allow contact between the sufentanil and a physiological fluid of a mammal into which the device is implanted to thereby release sufentanil in vivo from the device into the mammal.

Some embodiments include a method of treating pain or opioid addiction for an extended duration after a single administration of sufentanil, comprising: implanting one or more devices described herein into the body of a human being; wherein sufentanil is released from the devices into the body of the human being in an amount of about 250 μg to about 300 μg of sufentanil per day for at least about 6 months.

Some embodiments include a method of providing a constant serum concentration of sufentanil for an extended duration comprising: implanting one or more devices described herein into the body of a human being; wherein sufentanil is released from the devices into the body of the human being; and wherein the method provides a serum concentration of sufentanil in the human being in the range of about 10 μg/mL to about 50 μg/mL for at least about 6 months.

Some embodiments include implanting one or more devices described herein into a mammal or human body to treat pain.

Some embodiments include implanting one or more devices described herein into a mammal or human body to treat opioid addiction or dependency.

### DETAILED DESCRIPTION

The implantable devices described herein may be implanted into any mammal, including a human. When the device is implanted, it may continuously release sufentanil in vivo over an extended duration. This may improve compliance with drug dosing regimens and reduce abuse potential because a physician implants the device. These devices may also provide more constant blood levels of sufentanil than injectable, sublingual or transdermal dosage forms, and may thus minimize side effects.

Because of sufentanil's high potency, a therapeutic effect may be provided over an extended period of time using only very small quantities of sufentanil. This may allow smaller and/or fewer devices to be used while still providing extended release of sufentanil. This may provide greater flexibility for incorporating sufentanil into a polymeric device suitable for implantation.

Generally, an implantable device may comprise sufentanil and a solid biocompatible polymer matrix. The implantable devices described herein do not require external medical equipment such as an intravenous (IV) system, to release the drug. Nor do these implantable devices require an internal or external mechanical pump.

The shape of the devices may be modified according to where the device is implanted and other factors. In some embodiments, devices may be disk shaped, square or rectangular chip-shaped, cylindrical, square or rectangular rod-shaped (e.g. the cross-section of the rod is square or rectangular), etc. Shapes may be altered by varying the shape of the die in the extruder, cutting the extruded material, or by injecting extruded or mixed material into a mold. The above descriptions of shapes may be approximations, and the actual devices may deviate significantly from the ideal shape as long as the shape is recognizable.

Devices may be sized to provide the desired amount of drug according to the loading of the drug. In some embodiments, the device has a diameter of about 0.5 mm to about 5 mm, about 2 mm to about 10 mm, about 2 mm to about 5, mm, about 1 mm to about 2 mm, about 2 mm to about 3 mm, about 3 mm to about 4 mm, or about 4 mm to about 5 mm. In some embodiments, the device may be rigid because the polymer is rigid and the diameter is too large for the device to be flexible.

In some embodiments, the device has a length of about 1 cm to about 10 cm, about 1 cm to about 5 cm, about 1 cm to about 2 cm, about 2 cm to about 3 cm, about 3 cm to about 4 cm, or about 4 cm to about 5 cm. In some embodiments, the device may have a mass of about 1 mg to about 10 g, about 10 mg to about 5 g, or about 25 mg to about 1000 mg, about 20 mg to about 50 mg, about 50 mg to about 100 mg, about 100 mg to about 150 mg, about 150 mg to about 200 mg, or about 50 mg to about 200 mg. In some embodiments, the device may have volume of about 0.01 mL to about 2 mL, about 0.05 mL to about 1 mL, about 0.05 mL to about 0.1 mL, about 0.1 mL to about 0.15 mL, about 0.15 mL to about 0.2 mL, about 0.2 mL to about 0.3 mL, or about 0.05 mL to about 0.3 mL.

Multiple implantable devices may be used. The size of the device and the number of devices implanted may depend upon the rate and duration of the sustained release desired. In some embodiments, the number of devices implanted into a human being may be 1, 2, 3, 4, 5, 6, 7, 8, 9, 10, 11, 12, 13, 14, or 15. In some embodiments, the total weight of all devices implanted is about 60 mg to about 1 g.

For delivery of sufentanil to a human being for about 6 months, 1 or 2 devices may be implanted, wherein the total weight of the devices may be about 20 mg to about 150 mg. For delivery of sufentanil to a human being for about 12 months, 1, 2, 3, or 4 devices may be implanted, wherein the total weight of the devices may be about 40 mg to about 300 mg. For delivery of sufentanil to a human being for about 18 months, 1, 2, 3, 4, 5, or 6 devices may be implanted, wherein the total weight of the devices may be about 60 mg to about 450 mg. For delivery of sufentanil to a human being for about 24 months, 1, 2, 3, 4, 5, 6, 7, or 8 devices may be implanted, wherein the total weight of the devices may be about 80 mg to about 600 mg.

In some embodiments the number of devices implanted is 1, 2, 3, 4, 5, 6, 7, 8, 9, 10, 11, or 12 wherein each device has a weight of about 50 mg to about 100 mg. In some embodiments, the number of devices implanted is 2, 3, or 4.

While there are many ways that an implantable device may be fabricated, in some embodiments the device is prepared by a method comprising extruding a mixture of the polymer matrix and the sufentanil. The polymer and/or the sufentanil may be ground, mixed, and/or melted before extrusion. After extrusion, the material may be cut or injected into a mold. Methods that may be used include those described in U.S. Pat. No. 7,736,665, incorporated by references herein in its entirety.

Implantable devices described herein may be used to treat opioid addiction and pain, including any pain condition which may require administration of an analgesic substance, e.g., postoperative pain, cancer pain, arthritic pain, lumbosacral pain, musculoskeletal pain, neuropathic pain, etc. This list, however, should not be interpreted as exhaustive. Generally, when a device is implanted subcutaneously, it may provide systemic drug delivery, so that it may relieve all types of pain the recipient may be suffering, and not just pain localized to a particular area, organ, or tissue of the body. To treat opioid addiction or pain includes diagnosis, cure, mitigation, treatment, or prevention of opioid addiction or pain in a mammal or human being.

Implantable devices are administered by implantation in an individual, generally by a physician or another health care provider. The device may be implanted subcutaneously in any of a variety of sites of the body, such as the upper arm, the back, the abdomen, etc. Multiple implantable devices may be administered, and the number of devices is one way that the dosage of sufentanil may be controlled. Furthermore, the length of time during which sufentanil is administered may be extended by re-implanting additional implantable devices in an individual receiving treatment before or after serum levels of sufentanil begin to decline, to maintain sufentanil at the desired level.

The polymer matrix of the devices may comprise a plurality of pores for the release of the drug. The pores may be formed by incorporation of sufentanil into the polymeric matrix. When a device is implanted, the pores in the device may allow contact between the sufentanil and a physiological fluid of a mammal or a human being. A physiological fluid includes any fluid that is naturally in the body of the mammal or human being, such as blood or interstitial fluid. When sufentanil comes in contact with the physiological fluid through the pores, part of the sufentanil may dissolve or disperse in the physiological fluid to provide in vivo release of sufentanil from the device into the systemic circulation. This may allow the sufentanil to come in contact with areas of the body where pain relief is needed. In some embodiments, the sufentanil may be circulated in the bloodstream.

An implantable device may provide a near constant amount of sufentantil, similar to an IV drip, but without the need for additional any equipment or personnel. Some devices have an initial burst release of sufentanil immediately following implantation, and after the burst may release a substantially constant amount of sufentanil. The burst period may be avoided or reduced by prewashing the device in a solvent or solution in which the sufentanil is soluble such as water, saline, or an organic solvent such as ethanol. The desired dosage rate may depend upon factors such as the underlying condition for which sufentanil is being administered, and the physiology of a particular patient.

The release rate of sufentanil can be altered by modifying parameters such as the percent drug loading, porosity of the matrix, structure of the implantable device, the hydrophobicity of the matrix, or the number of devices. A hydrophobic coating or a biodegradable coating may be placed over at least a portion of the device to further regulate the rate of release.

When implanted, the device or devices may release a therapeutically effective amount of sufentanil for an extended period of time after a single administration of sufentanil. A single administration of sufentanil includes administration of one or more devices or one or more dosage forms at substantially the same time, including for example, a single visit to a physician.

An extended period of time includes any period of time significantly longer than the amount of time an oral dosage form may provide a therapeutically effective amount of sufentanil after administration. In some embodiments, the devices may provide a therapeutically effective amount of sufentanil for at least about 1 month, at least about 3 months, at least about 6 months, at least about 9 months, at least about 1 year, at least about 15 months, at least about 18 months, at least about 2 years, about 1 month to about 3 years, about 3 months to about 2 years, about 3 months to about 6 months, about 6 months to about 9 months, about 9 months to about 1 year, about 1 year to about 15 months, about 15 months to about 18 months, about 18 months to about 21 months, about 21 months to about 24 months, about 3 months to about 1 year, about 6 months to about 1 year, or about 1 year to about 2 years. The extended delivery provided by some devices may reduce the need for external medical equipment and personnel associated with intravenous methods.

The amount of sufentanil released in vivo from the device or devices may vary according to the circumstances. In some embodiments, the device or devices may release sufentanil in vivo at a rate of about 20 μg/day to about 1 mg/day, about 100 pg/day to about 500 μg/day, about 100 μg/day to about 200 μg/day, about 200 μg/day to about 300 μg/day, about 300 μg/day to about 400 μg/day, or about 400 μg/day to about 500 μg/day. In some embodiments, the ratio of the average to the standard deviation of the amount of sufentanil released each day may be less than about 1, about 0.5, about 0.3, about 0.2, or about 0.1 for a time period of at least 1 month, at least about 2 months, at least about 3 months, or about 1 months to about 6 months after the device or devices are implanted. Since some devices have the burst period, this time period of substantially constant release may begin some time later than implantation, such as about 1 month, about 2 months, or about 3 months after implantation.

In some embodiments, the device or devices may be configured to provide a human serum concentration of sufentanil of about 0.5 μg/mL to about 500 pg/mL, about 1 μg/mL to about 200 μg/mL, about 5 μg/mL to about 10 μg/mL, or about 10 μg/mL to about 30 μg/mL, about 30 μg/mL to about 50 μg/mL, about 50 μg/mL to about 80 μg/mL, or about 5 μg/mL to about 80 μg/mL. Some devices may provide a substantially constant serum concentration of sufentanil after a burst period. In some embodiments, the ratio of the average to the standard deviation of the serum concentration taken daily may be less than about 1, about 0.5, about 0.3, about 0.2, or about 0.1 for a time period of at least 1 month, at least about 2 months, at least about 3 months, or about 1 month to about 6 months after the device is implanted. Since some devices have the burst period, this time period of substantially constant serum concentration may begin some time later than implantation, such as about 1 month, about 2 months, or about 3 months after implantation.

Sufentanil has the structure shown below.

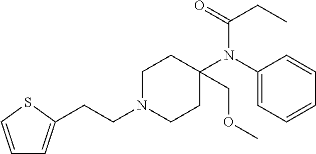 [SMILES: CCC(=O)N(c1ccccc1)C1(COC)CCN(CCc2cccs2)CC1]

In an implantable device, sufentanil may be present as a free base or in any pharmaceutically acceptable salt form. In some embodiments, the sufentanil in the device is present as a free base, or as a salt, such as a citrate, lactate, tartrate, maleate, succinate, glycolate, phosphate, sulfate, or hydrochloride salt.

Some devices may use other opioids as a substitute for, or in conjunction with, sufentanil. Examples may include, but are not limited to, alfentanil, allyiprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethyhnethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papveretum, pentazocine, phenadoxone, phenomorphan, phenazocine, phenoperidine, piminodine, piritramide, propheptazine, promedol, properidine, propoxyphene, tilidine, tramadol and their pharmaceutically acceptable salts.

The amount of sufentanil in a device may vary depending upon circumstances such as the amount of sufentantil needed for the treatment, the duration that the sufentanil will need to be delivered, and the number of devices to be administered. In some embodiments, the device or devices implanted in a human being comprise about 1 mg to about 1000 mg, about 10 mg to about 500 mg, about 20 mg to about 50 mg, about 50 mg to about 80 mg, about 80 mg to about 120 mg, about 120 mg to about 150 mg, about 150 mg to about 200 mg, or about 20 mg to about 200 mg of sufentanil, such as sufentanil citrate or sufentanil free base.

The proportion of the sufentanil in the device may vary. Lower drug loading of the device or devices may allow slower release of the drug. Conversely, greater loading of the device or devices may allow more rapid release of the drug. This may be because a greater loading of the drug creates a larger number of pores in a device, thus increasing the release rate of the drug. In some embodiments, the sufentanil is about 10% to about 20%, about 30% to about 40%, about 40% to about 50%, about 60% to about 70%, about 70% to about 80%, about 80% to about 90%, or about 50% to about 75% of a device based upon weight.

In some embodiments, the rate of drug delivery in a device may be increased by including a water soluble filler to increase the number of pores in the extruded device. Any water soluble compound or salt that can be tolerated in a human or mammal body may be used. Examples of useful fillers may include, but are not limited to, sugars, such as glucose, mannose, galactose, lactose, fructose, sucrose, etc.; amino acids, such as glycine, alanine, valine, leucine, isoleucine, methionine, proline, phenylalanine, tryptophan, asparagine, glutamine, serine, threonine, aspartic acid, glutamic acid, tyrosine, cysteine, lysine, arginine, histidine, etc.; salts such as sodium chloride, phosphate salts, sulfate salts, etc.; organic acids such as citric acid, ascorbic acid, glycolic acid, etc; etc.

A biocompatible polymer matrix may be any polymeric material suitable for use in an mammal, including a human being. A biodegradable polymer matrix may disintegrate during delivery of the sufentanil so that it may not need to be removed after use. Examples of biodegradable polymer may include, but are not limited to: polyesters, polyorthoesters, polyphosphoesters, polycarbonates, polyanhydrides, polyphosphazenes, polyoxalates, polyaminoacids, polyhydroxyalkanoates, polyethyleneglycol, polyvinylacetate, polyhydroxyacids, polyanhydrides, copolymers and blends thereof, and the like. In some embodiments, a biodegradable polymer may be a co-polymer of lactic and glycolic acid.

A nondegradable polymer may also be used, including any biocompatible polymer that is substantially intact after release of sufentanil by a device is substantially complete. A nondegradable polymer may provide a more constant release of sufentanil than a biodegradable polymer. Examples of nondegradable polymers may include, but are not limited to: ethylene vinyl acetate copolymer (EVA), silicone, hydrogels such as crosslinked poly(vinyl alcohol) and poly(hydroxy ethylmethacrylate), acyl substituted cellulose acetates and alkyl derivatives thereof, partially and completely hydrolyzed alkylene-vinyl acetate copolymers, polyvinyl chloride, homo- and copolymers of polyvinyl acetate, polyethylene, polypropylene, crosslinked polyesters of acrylic acid and/or methacrylic acid, alkyl acrylates such as methyl methacrylate or methyl acrylate, polyacrylic acid, polyalkacrylic acids such as polymethacrylic acid, polyvinyl alkyl ethers, polyvinyl fluoride, polytetrafluoroethylene, polycarbonate, polyurethane, polyimide, polysulphones, polystyrene, styrene acrylonitrile copolymers, poly(ethylene oxide), poly(alkylenes), poly(vinyl imidazole), poly(esters), poly(ethylene terephthalate), polyphosphazenes, and chlorosulphonated polyolefins, and combinations thereof.

In some embodiments, the polymer matrix comprises an ethylene vinyl acetate copolymer. In some embodiments, the ethylene vinyl acetate copolymer comprises about 20% to about 50%, about 30% to about 40%, or about 30% to about 35% vinyl acetate by weight.

Any of the above polymers may also be used to coat a device in order to decrease the rate of release of the drug.

Some embodiments include a kit for use in opioid addiction or pain. The kits comprise at least one device described herein and instructions providing information regarding implantation of the device for treatment of opioid addiction or pain.

### EXAMPLE 1

A mixture of 50% sufentanil citrate and 50% ethylene vinyl acetate (having 33% vinyl acetate) by weight is extruded through a die having a diameter around 2 mm. The temperature of the extruder and the die are about 200 ° F. to about 250 ° F. The extruded mixture is cut into rods having a length of about 25 mm.

### EXAMPLE 2

The procedure of Example 1 is repeated, except that the mixture is 60% sufentanil citrate and 40% ethylene vinyl acetate.

### EXAMPLE 3

The procedure of Example 1 is repeated, except that the mixture is 70% sufentanil citrate and 30% ethylene vinyl acetate.

### EXAMPLE 4

One extruded rod of Example 1 is implanted into an upper arm of a human patient to provide extended delivery of sufentanil to the patient.

### EXAMPLE 5

One extruded rod of Example 2 is implanted into an upper arm of a human patient to provide extended delivery of sufentanil to the patient.

### EXAMPLE 6

One extruded rod of Example 3 is implanted into an upper arm of a human patient to provide extended delivery of sufentanil to the patient.

### EXAMPLE 7

Two extruded rods of Example 1 are implanted into an upper arm of a human patient to provide extended delivery of sufentanil to the patient.

### EXAMPLE 8

Two extruded rods of Example 2 are implanted into an upper arm of a human patient to provide extended delivery of sufentanil to the patient.

### EXAMPLE 9

Two extruded rods of Example 3 are implanted into an upper arm of a human patient to provide extended delivery of sufentanil to the patient.

### EXAMPLE 10

Three extruded rods of Example 1 are implanted into an upper arm of a human patient to provide extended delivery of sufentanil to the patient.

### EXAMPLE 11

Three extruded rods of Example 2 are implanted into an upper arm of a human patient to provide extended delivery of sufentanil to the patient.

### EXAMPLE 12

Three extruded rods of Example 3 are implanted into an upper arm of a human patient to provide extended delivery of sufentanil to the patient.

### EXAMPLE 13

Four extruded rods of Example 1 are implanted into an upper arm of a human patient to provide extended delivery of sufentanil to the patient.

### EXAMPLE 14

Four extruded rods of Example 2 are implanted into an upper arm of a human patient to provide extended delivery of sufentanil to the patient.

### EXAMPLE 15

Four extruded rods of Example 3 are implanted into an upper arm of a human patient to provide extended delivery of sufentanil to the patient.

Although the claims have been described in the context of certain preferred embodiments and examples, it will be understood by those skilled in the art that the scope of the claims extend beyond the specifically disclosed embodiments to other alternative embodiments and/or uses and obvious modifications and equivalents thereof.

1-20. (canceled)

21. A composition comprising:
(a) a nondegradable polymer or a biocompatible polymer matrix; and
(b) sufentanil or a pharmaceutically acceptable salt thereof.

22. The composition of claim 21, wherein the composition further comprises:
(c) a water soluble filler.

23. The composition of claim 22, wherein the composition essentially consists of:
(a) a nondegradable polymer or a biocompatible polymer matrix;
(b) sufentanil or a pharmaceutically acceptable salt thereof; and
(c) a water soluble filler.

24. The composition of claim 21, wherein the nondegradable polymer is an ethylene vinyl acetate copolymer, a silicone, a hydrogel, an acyl substituted cellulose acetate, an alkyl derivative of an acyl substituted cellulose acetate, a partially hydrolyzed alkylene-vinyl acetate copolymer, a completely hydrolyzed alkylene-vinyl acetate copolymer, a polyvinyl chloride, a homo-polymer of polyvinyl acetate, a copolymer of polyvinyl acetate, a polyethylene, a polypropylene, a crosslinked polyester of acrylic acid, a crosslinked polyester of methacrylic acid, a crosslinked polyester of acrylic acid and methacrylic acid, an alkyl acrylate, a polyacrylic acid, a polyalkacrylic acid, a polyvinyl alkyl ether, a polyvinyl fluoride, a polytetrafluoroethylene, a polycarbonate, a polyurethane, a polyamide, a polysulphone, a polystyrene, a styrene acrylonitrile copolymer, a poly(ethylene oxide), a poly(alkylene), a poly(vinyl imidazole), a poly(ester), a poly(ethylene terephthalate), a polyphosphazene, or a chlorosulphonated polyolefin, or a combination thereof.

25. The composition of claim 24, wherein:
the hydrogel is a crosslinked poly(vinyl alcohol) or a poly(hydroxy ethylmethacrylate);
the alkyl acrylate is a methyl methacrylate or a methyl acrylate; or the polyalkacrylic acid is a polymethacrylic acid.

26. The composition of claim 21, wherein the biocompatible polymer matrix is a polyester, a polyorthoester, a polyphosphoester, a polycarbonate, a polyphosphazene, a polyoxalate, a polyaminoacid, a polyhydroxyalkanoate, a polyethyleneglycol, a polyvinylacetate, a polyhydroxyacid, or a polyanhydride, or a copolymer or blend thereof.

27. The composition of claim 22, wherein the water soluble filler is a sugar, an amino acid, a salt or an organic acid.

28. The composition of claim 27, wherein:
the sugar is a glucose, a mannose, a galactose, a lactose, a fructose or a sucrose;
the amino acid is a glycine, an alanine, a valine, a leucine, an isoleucine, a methionine, a proline, a phenylalanine, a tryptophan, an asparagine, a glutamine, a serine, a threonine, an aspartic acid, a glutamic acid, a tyrosine, a cysteine, a lysine, an arginine, or a histidine;
the salt is a sodium chloride, a phosphate or a sulfate; or the organic acid is a citric acid, an ascorbic acid or a glycolic acid.

29. The composition of claim 24, wherein the nondegradable polymer is an ethylene vinyl acetate copolymer.

30. The composition of claim 29, wherein the ethylene vinyl acetate copolymer comprises about 20% to about 50% vinyl acetate by weight.

31. The composition of claim 21, wherein the nondegradable polymer is an ethylene vinyl acetate copolymer, and the opioid is sufentanil or a pharmaceutically acceptable salt thereof.

32. The composition of claim 31, wherein the composition comprises about 50% to about 70% sufentanil, or a pharmaceutically acceptable salt thereof, by weight.

33. The composition of claim 31, wherein the composition comprises about 30% to about 50% ethylene vinyl acetate copolymer by weight.

34. The composition of claim 33, wherein the ethylene vinyl acetate copolymer comprises about 33% vinyl acetate by weight.

35. The composition of claim 21, wherein the composition is a solid mixture.

36. The composition of claim 21, wherein the opioid is sufentanil or sufentanil citrate.

37. The composition of claim 21, wherein the composition is a solid mixture essentially consisting of:
(a) about 30% to about 50% by weight of an ethylene vinyl acetate copolymer comprising about 33% vinyl acetate by weight; and
(b) about 50% to about 70% by weight sufentanil or sufentanil citrate.

38. The composition of claim 23, wherein the composition is a solid mixture essentially consisting of:
(a) about 30% to about 50% by weight of an ethylene vinyl acetate copolymer comprising about 33% vinyl acetate by weight;
(b) about 50% to about 70% by weight sufentanil or sufentanil citrate; and
(c) a water soluble filler.

39. An implantable device, wherein the device comprises the composition of claim 21.
